SEQUENCE LISTING (3 sequences)

SEQ ID NO: 1 - protein, 24 aa, mouse
HPFTEDDAVDPNDSDIDPESRETD

SEQ ID NO: 2 - protein, 24 aa, Homo sapiens
RPFTEDDAADPNDSDIDPESRETD

SEQ ID NO: 3 - protein, 24 aa, chicken
RPFTEDDAFDPNDSDIDPESRETD

(19) United States
(12) Patent Application Publication
(10) Pub. No.: US 2004/0096851 A1
(43) Pub. Date: May 20, 2004

(54) ABCA1 PEST domain-related compositions and methods

(76) Inventors: Nan Wang, Flushing, NY (US); Alan R. Tall, Creskil, NJ (US); David L. Silver, New York, NY (US)

Correspondence Address: Cooper and Dunham LLP, 1185 Avenue of the Americas, New York, NY, 10036, US

(21) Appl. No.: 10/425,406

(22) Filed: Apr. 28, 2003

Related U.S. Application Data
(60) Provisional application No. 60/375,656, filed on Apr. 26, 2002.

(51) Int. Cl. C12Q 1/68; G01N 33/53; C07H 21/04; C07K 14/705

(52) U.S. Cl. 435/6; 435/69.1; 435/320.1; 435/325; 530/350; 536/23.5

(57) ABSTRACT

This invention provides an isolated ABCA1 protein having a mutation within its PEST domain. This invention also provides related nucleic acids, vectors, host cells, pharmaceutical compositions and articles of manufacture. This invention further provides methods for determining whether a protease inhibitor increases the rate of cholesterol efflux from a cell, for increasing ABCA1-mediated cholesterol efflux from a cell, for treating atherosclerosis in a subject, and for increasing HDL formation in a subject.

This invention was made in part with support under United States Government NIH Grant HL58948. Accordingly, the United States Government has certain rights in this invention.

This application claims priority of U.S. Serial No. 60/375,656, filed Apr. 26, 2002, the contents of which are hereby incorporated by reference.

Throughout this application various publications are referenced by number. Full citations for these publications can be found at the end of the specification. The disclosures of these publications in their entireties are hereby incorporated by reference into the application in order to more fully describe the state of the art known as of the date of the invention claimed herein.

### BACKGROUND OF THE INVENTION

Atherosclerosis, the leading cause of morbidity and death in industrialized societies, is initiated by the deposition of lipoprotein cholesterol in the artery wall. After retention and modification in arteries, atherogenic lipoproteins are taken up by macrophages, giving rise to cholesterol-engorged foam cells (1). The progression of atherosclerosis can be delayed or reversed by removal of cholesterol from foam cells in a process involving high-density lipoproteins (HDL) (2). Currently, the major therapeutic approach to atherosclerosis involves the lowering of blood levels of atherogenic lipoproteins.

Intense interest has recently centered on the possibility that increasing cholesterol efflux from foam cells via HDL could represent a novel approach to treating this disease (3).

In Tangier Disease very low plasma HDL levels are associated with macrophage foam cell accumulation in various organs such as the spleen or tonsils and there is an excess of atherosclerotic cardiovascular disease (4). Tangier Disease is caused by mutations in the ATP-binding cassette transporter, ABCA1 (5, 6, 7). ABCA1 mediates phospholipid and cholesterol efflux to free apolipoproteins, such as apoA-I, forming nascent HDL (8). Nascent HDL particles formed by the interaction of apoA-I with ABCA1 on hepatocytes, macrophages and other cells, mature in the bloodstream and their cholesterol is eventually returned to the liver. Thus, the up-regulation of ABCA1 expression may provide a key to the promotion of foam cell cholesterol efflux and HDL formation (9).

The cellular expression of ABCA1 is highly regulated. In many cells, such as macrophages, ABCA1 protein is undetectable in the basal state, but expression is markedly increased with cholesterol loading, as a result of LXR/RXR-mediated transcription (9, 10). However, turnover of ABCA1 protein in macrophages is rapid (11) and often, the increase of ABCA1 protein is not proportionate to the increase of ABCA1 mRNA (12). Together, these suggest that ABCA1 turnover plays a major role in regulation of ABCA1 function even though the mechanisms are poorly understood.

### SUMMARY OF THE INVENTION

This invention provides for an isolated ABCAL protein having a mutation within its PEST domain, an isolated nucleic acid encoding an ABCA1 protein having a mutation within its PEST domain, and a vector comprising the instant nucleic acid.

This invention further provides a host-vector system comprising a host cell having therein the instant expression vector.

This invention further provides an isolated cell comprising an ABCA1 protein having a mutation within its PEST domain of the protein, which protein has a decreased rate of turnover in the cell.

This invention further provides a method for determining whether a protease inhibitor reduces the turnover rate of ABCA1 protein in a cell, comprising the steps of

(a) contacting the cell with the protease inhibitor under physiological conditions;

(b) determining the rate of ABCAL protein turnover in the cell; and

(c) comparing the rate of turnover so determined with a known standard, thereby determining whether the protease inhibitor reduces the ABCA1 protein turnover rate in the cell.

This invention further provides a method for determining whether a protease inhibitor increases the rate of cholesterol efflux from a cell, comprising the steps of

(a) contacting the cell with the protease inhibitor under physiological conditions;

(b) determining the rate of cholesterol efflux from the cell; and

(c) comparing the rate of cholesterol efflux so determined with a known standard, thereby determining whether the protease inhibitor increases the rate of cholesterol efflux from the cell.

This invention further provides a method for increasing ABCA1-mediated cholesterol efflux from a cell comprising the step of inhibiting the PEST-mediated proteolysis of ABCA1 protein in the cell.

This invention further provides a method for treating a subject afflicted with atherosclerosis comprising administering to the subject an agent that inhibits the PEST-mediated proteolysis of ABCA1 protein in the subject's macrophages.

This invention further provides a method for increasing the formation of HDL in a subject comprising administering to the subject an agent that inhibits the PEST-mediated proteolysis of ABCA1 protein in the subject's cells.

This invention further provides for a pharmaceutical composition comprising an agent that inhibits the PEST-mediated proteolysis of ABCA1 protein in a cell, and a pharmaceutically acceptable carrier.

Finally, this invention provides for an article of manufacture comprising a packaging and a pharmaceutical agent, wherein (a) the pharmaceutical agent inhibits the PEST-mediated proteolysis of ABCA1 protein in a cell, and (b) the packaging comprises a label indicating the use of the agent for treating atherosclerosis in a subject.

### BRIEF DESCRIPTION OF THE FIGURES

FIGS. 1A-1H. PEST-regulated function of ABCA1

FIG. 1A shows a schematic model of ABCA1 showing the location and amino acid sequence of PEST and alignment among mouse (m), human (h) and chicken (c) ABCA1. The PEST score is +16.42, as determined by PESTFIND. FIG. 1B shows levels of total (lower panel) or cell surface (upper panel, biotinylated) ABCA1 or ABCA1delPEST in transiently transfected HEK293 cells. FIG. 1C shows levels of total (lower panel) or ubiquitinated (upper panel) ABCA1 or ABCA1delPEST with or without lactacystin (20 μM) for 3 h. DNA dose-dependent ABCA1- or ABCA1delPEST-mediated cellular cholesterol efflux (FIG. 1D), phospholipid efflux (FIG. 1G) or apoA-T cell association (FIG. 1H). FIG. 1E shows the average of duplicate determination of ABCA1 protein mass in cholesterol efflux assay. FIG. 1F shows cholesterol efflux efficiency determined by percentage efflux over ABCA1 protein mass.

FIGS. 2A-2E. Calpain protease catalyzes PEST-dependent degradation of ABCA1

Levels of ABCA1 or ABCA1delPEST in 293 cells with or without calpeptin (30 μg/ml) for 3 hours (FIG. 2A) by Western analysis. The susceptibility to exogenously added p-calpain of ABCA1 or ABCA1delPEST metabolically labeled by [35S] methionine in digitonin-permeabilized 293 cells with (FIG. 2C) or without (FIG. 2B) pretreatment of apoA-I (10 μg/ml) for 3 hours. Levels of ABCA1 in the absence or presence of calpeptin in mouse peritoneal macrophages (FIG. 2D) or mouse primary hepatocytes (FIG. 2E).

FIGS. 3A-3F. Effect of apoA-I on ABCA1 protein level FIG. 3A shows wild type ABCA1 (Wt) and ABCA1delPEST (PS) transiently transfected 293 cells were incubated with or without apoA-I (10 μg/ml) in 0.2% BSA/DMEM for 3 hours. The cells were then lysed with RIAP buffer containing protease inhibitors and calpeptin. Equal aliquots of total cell lysis were loaded for ABCA1 western blotting. The membrane was re-probed with anti-actin antibody. In FIGS. 3B-3D, mouse peritoneal macrophages were treated overnight with 50 μg/ml AcLDL and LXR/RXR ligands 22(R)-hydroxycholesterol and 9-cis retinoic acid (both in 10 μM) to induce ABCA1 level. After wash, the cells were incubated with different apoA-I concentrations for 3 hours (FIG. 3B); or with 10 μg/ml apoA-I for different periods (FIG. 3C) at 37° C. in 0.2° BSA/DMEM. The cells were then scrapped and lysed with RIAP buffer. Equal amount of protein (30 μg per lane) was loaded for ABCA1 western blotting. In FIG. 3D, after 3 hours incubation with apoA-I, the macrophages were first cell surface biotinylated and then precipitated with streptavidin-conjugated beads. The beads were eluted with SDS-PAGE loading buffer and equal aliquots were loaded for ABCA1 western blotting. The membranes were re-probed with anti-integrin antibody. In FIG. 3E, cells were treated with or without the AcLDL and LXR/RXR ligands and effect of apoA-1 on ABCA1 level was determined as per FIGS. 3B, 3D. In FIG. 3F, primary hepatocytes were incubated with or without apoA-I (10 μg/ml) for 3 hours. 30 μg of total cell lysis were loaded for ABCA1 western blotting.

FIGS. 4A-4E. ApoA-I mediated phospholipid efflux on ABCA1 protein level

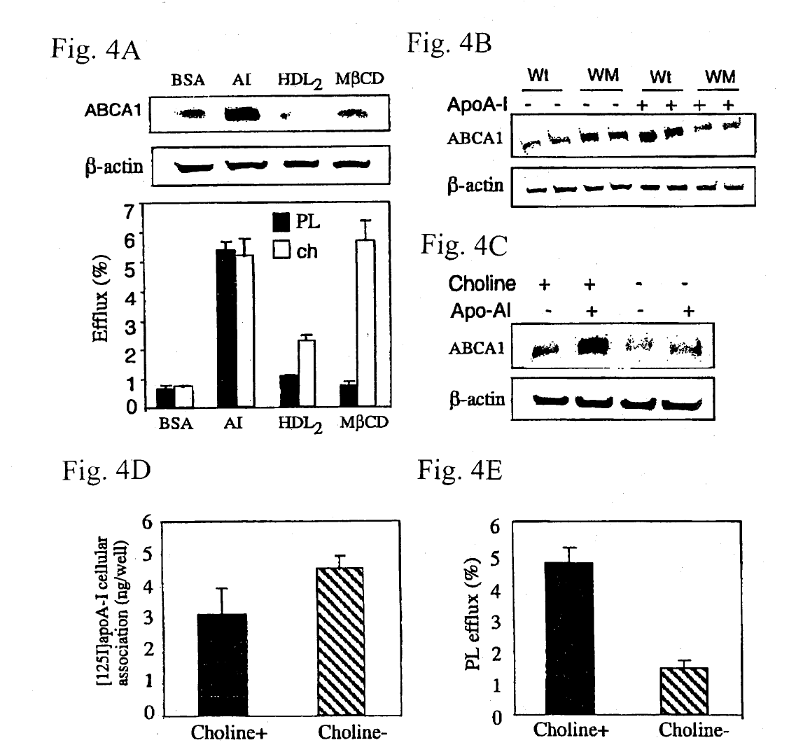

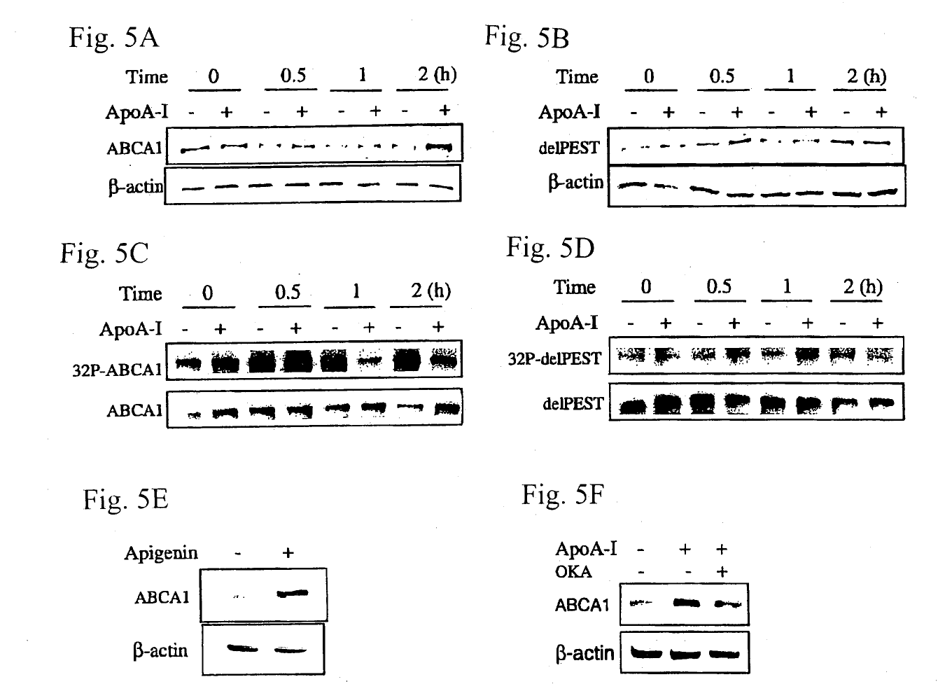

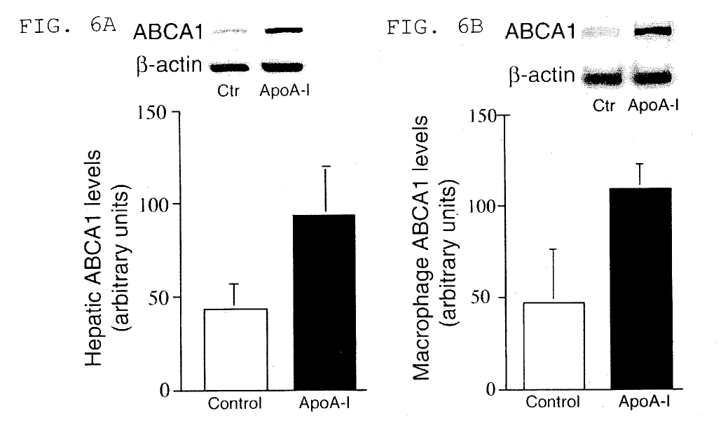

In FIG. 4A, mouse peritoneal macrophages were incubated with control (BSA), 10 μg/ml apoA-1 and 50 μg/ml HDL2 for 3 hours, or first with 1 mM methy-β-cyclodextrin (MβCD) for 5 min to deplete cholesterol, then with control medium, for 3 hours. After incubation the cells were lysed and 30 μg total protein were loaded for western blotting. Phospholipid and cholesterol efflux was carried out with the same treatment conditions. In FIG. 4B, wild type ABCA1 (Wt) and Walker-mutant ABCA1 (WM) transiently transfected 293 cells were incubated with or without apoA-I (10 μg/ml) for 3 hours and total cell lysates were run for ABCA1 western blotting. In FIG. 4C, mouse peritoneal macrophages were culture in 10% FBS/DMEM or dialyzed 10% FBS/choline-free DMEM for 2 days. After overnight induction of ABCA1 by AcLDL and LXR/RXR ligands, the cells were incubated with or without apoA-I for 3 hours. 30 μg of total cell lysis were run for ABCA1 western blotting. In FIG. 4D, cells were treated as in FIG. 4C and incubated with 0.5 μg/ml 125-idionated apoA-I for 1 hour at 37° C. After washing, the cells were lysed and total radio counts were determined. In FIG. 4E, mouse peritoneal macrophages were ³H-choline (0.5 μCi/ml) labeled for 8 hours immediately after isolation. After wash, the cells were incubated with 10% FBS/DMEM or choline-free medium for 2 days. After overnight induction of ABCA1, phospholipid efflux to apoA-I was done for 2 hours as described above.

FIGS. 5A-5F. ApoA-I inhibits degradation of ABCA1 by decreasing PEST-dependent phosphorylation of ABCA1

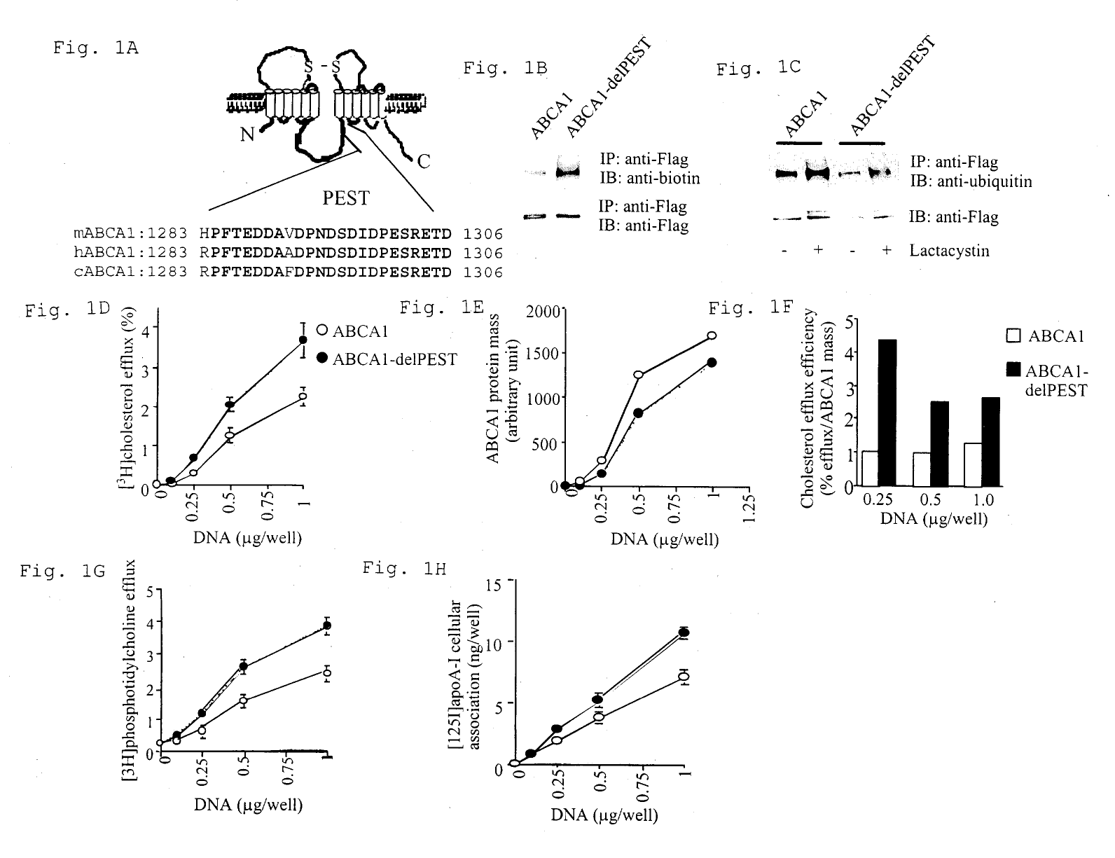

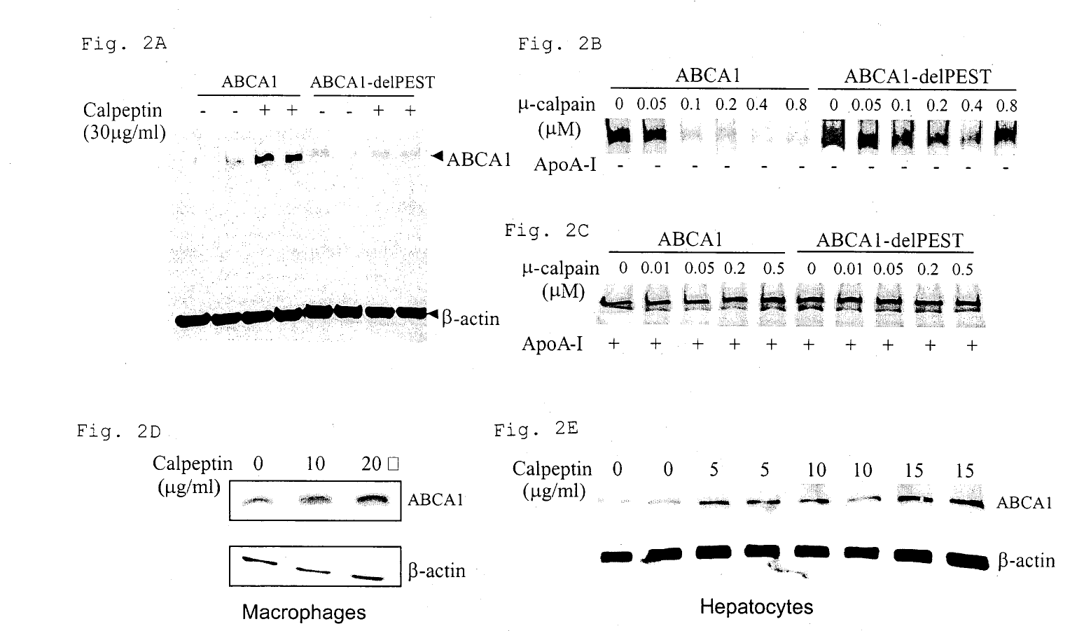

Effect of apoA-I (10 μg/ml) treatment on ABCA1 or ABCA1delPEST; ABCA1 (FIG. 5A) or ABCA1delPEST (FIG. 5B) total protein mass; ABCA1 phosphorylation (FIG. 5C, upper panel) and protein mass (FIG. 5C, lower panel); ABCA1delPEST phosphorylation (FIG. 5D, upper panel) and protein mass FIG. 1 (5D, lower panel). (FIG. 5E) Effect of apigenin (40 μM) treatment for 3 hours on ABCA1 protein levels in mouse peritoneal macrophages; (FIG. 5F) Okadaic acid reverses the increase of ABCA1 by apoA-I in macrophages.

FIGS. 6A and 6B. ApoA-I injection increases ABCA1 protein in hepatocytes and macrophages in mice

(FIG. 6A) Hepatic ABCAL levels in mice 4 hours after intravenous injection of apoA-I (20 mg/kg body weight) or albumin as control (Ctr). The bar graph represents quantification of ABCA1 protein levels normalized against β-actin (n=5 for each group, P<0.01). (FIG. 6B) Macrophage ABCA1 levels in mice 4 hours after apoA-I injection (n=5 for apoA-T group and n=3 for control group, P<0.006).

### DETAILED DESCRIPTION OF THE INVENTION

Definitions

As used in this application, except as otherwise expressly provided herein, each of the following terms shall have the meaning set forth below.

“ABCA1 protein” shall mean ATP-binding cassette A1 protein.

“Administering” shall mean delivering in a manner, which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

“Host cells” include, but are not limited to, bacterial cells, yeast cells, fungal cells, insect cells, and mammalian cells. Mammalian cells can be transfected by methods well-known in the art such as calcium phosphate precipitation, electroporation and microinjection.

“Isolated”, with respect to ABCA1 protein, shall mean an ABCA1 protein-containing membrane fragment preparation or other suitable preparation wherein ABCA1 retains its natural function and is free from some or all of the other proteins in its native milieu.

“Mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells.

The terms “nucleic acid” and “polynucleotide” are used interchangeably herein, and each refers to a polymer of deoxyribonucleotides and/or ribonucleotides. The deoxyribonucleotides and ribonucleotides can be naturally occurring or synthetic analogues thereof.

“PEST” domain shall mean a domain within a protein enriched with respect to proline (P), glutamic acid (E), serine (S) and threonine (T). In one embodiment, PEST domain shall mean the sequence in human ABCA1 protein from about residue 1283 to about residue 1306.

“Pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer=s dextrose, and the like. Preservatives and other additives may also be present, such as, for example, antimicrobials, antioxidants, chelating agents, inert gases, and the like.

The term “physiological conditions” shall mean, with respect to a given cell, such conditions, which would normally constitute the cell's biochemical milieu. The cell's biochemical milieu includes, without limitation some or all the proteases to which the cell is normally exposed. Such conditions include, but are not limited, to in vivo conditions.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

“Subject” shall mean any animal, such as a mammal or a bird, including, without limitation, a cow, a horse, a sheep, a pig, a dog, a cat, a rodent such as a mouse or rat, a turkey, a chicken and a primate. In the preferred embodiment, the subject is a human being.

“Treating” shall include, without limitation, eliminating, reversing the course of, slowing the progression of, reducing the symptoms of, or otherwise ameliorating, a disease in a subject.

“Vector” shall mean any nucleic acid vector known in the art. Such vectors include, but are not limited to, plasmid vectors, cosmid vectors, and bacteriophage vectors.

Embodiments of the Invention

This invention provides an isolated ABCA1 protein having a mutation within its PEST domain.

In one embodiment, the protein is a human protein. In another embodiment, the mutation is selected from the group consisting of a point mutation, an insertion mutation and a deletion mutation. Preferably, the mutation is a deletion mutation.

This invention further provides an isolated nucleic acid encoding an ABCA1 protein having a mutation within its PEST domain. The nucleic acid can be DNA or RNA, and preferably DNA.

This invention further provides a vector comprising the instant nucleic acid. In one embodiment, the vector is an expression vector.

This invention further provides a host-vector system comprising a host cell having therein the instant expression vector. The cell can be a prokaryotic cell or an eukaryotic cell. In one embodiment, the cell is a mammalian cell.

This invention further provides an isolated cell comprising an ABCA1 protein having a mutation within its PEST domain of the protein, which protein has a decreased rate of turnover in the cell. This invention further provides a first method for determining whether a protease inhibitor reduces the turnover rate of ABCA1 protein in a cell, comprising the steps of

(a) contacting the cell with the protease inhibitor under physiological conditions;

(b) determining the rate of ABCA1 protein turnover in the cell; and

(c) comparing the rate of turnover so determined with a known standard, thereby determining whether the protease inhibitor reduces the ABCA1 protein turnover rate in the cell.

This invention further provides a second method for determining whether a protease inhibitor increases the rate of cholesterol efflux from a cell, comprising the steps of

(a) contacting the cell with the protease inhibitor under physiological conditions;

(b) determining the rate of cholesterol efflux from the cell; and

(c) comparing the rate of cholesterol efflux so determined with a known standard, thereby determining whether the protease inhibitor increases the rate of cholesterol efflux from the cell.

In one embodiment of the first and second methods, the cell is a human cell. In a further embodiment, the cell is a macrophage. In yet a further embodiment, the protease inhibitor is a calpain inhibitor.

This invention further provides a third method for increasing ABCA1-mediated cholesterol efflux from a cell comprising the step of inhibiting the PEST-mediated proteolysis of ABCA1 protein in the cell.

In one embodiment of the third method, the cell is a human cell. In another embodiment, the cell is a macrophage. In a further embodiment, the inhibiting comprises contacting the cell with a calpain inhibitor.

This invention further provides a fourth method for treating a subject afflicted with atherosclerosis comprising administering to the subject an agent that inhibits the PEST-mediated proteolysis of ABCA1 protein in the subject's macrophages. In one embodiment of the fourth method, treating the subject comprises stabilizing arterial plaque in the subject.

This invention further provides a fifth method for increasing the formation of HDL in a subject comprising administering to the subject an agent that inhibits the PEST-mediated proteolysis of ABCAL protein in the subject's cells.

In one embodiment of the fourth and fifth methods, the subject is a human. In another embodiment, the agent is a protease inhibitor. In a further embodiment, the protease inhibitor is a calpain inhibitor.

This invention further provides a pharmaceutical composition comprising an agent that inhibits the PEST-mediated proteolysis of ABCA1 protein in a cell, and a pharmaceutically acceptable carrier.

Finally, this invention provides an article of manufacture comprising a packaging and a pharmaceutical agent, wherein (a) the pharmaceutical agent inhibits the PEST-mediated proteolysis of ABCA1 protein in a cell, and (b) the packaging comprises a label indicating the use of the agent for treating atherosclerosis in a subject.

In one embodiment of the instant pharmaceutical composition or article, the agent is a protease inhibitor. In another embodiment, the protease inhibitor is a calpain inhibitor. In a further embodiment, the subject is a human.

This invention is illustrated in the Experimental Details section, which follows. This section is set forth to aid in an understanding of the invention but is not intended to, and should not be construed to, limit in any way the invention as set forth in the claims which follow thereafter.

Experimental Details

Part I: ABCA1 Degradation is Regulated by a PEST Sequence and is Mediated by Calpain Protease

Synopsis

Cholesterol-loaded macrophage foam cells are a central component of atherosclerotic lesions. ABCA1, the defective molecule in Tangier Disease, mediates the efflux of phospholipids and cholesterol from cells to apolipoproteins, reversing foam cell formation. A proline, glutamate, serine, threonine-rich (PEST) sequence in ABCA1 was identified and shown to regulate the cell surface concentration and activity of ABCA1. ABCA1 is a target of p-calpain protease degradation, an effect requiring the PEST sequence. In a novel form of positive feedback regulation, apoA-I mediates lipid efflux, phosphorylation of the PEST sequence is decreased and calpain degradation of ABCA1 is inactivated, leading to increased ABCA1 protein levels.

Discussion

It was shown that ABCA1 protein degradation is regulated by a PEST sequence and is mediated by calpain protease. In a novel form of positive feedback control, the interaction of ABCA1 with apoA-I leads to inhibition of calpain protease degradation.

Using metabolic labeling in transfected 293 cells, it was shown that ABCA1 protein turns over rapidly, with a half-life of approximately one hour (data not shown). Furthermore, treatment of cells with non-specific cysteine protease inhibitors (ALLN, MG132, leupeptin and E64) resulted in an increase in the half-life to 4-6 hrs and increased ABCA1 protein levels 2-4 fold. Many proteins that undergo rapid turnover contain sequences enriched in proline, glutamic acid, serine and threonine, called PEST sequences (13). Using the program PESTfind, a high probability, conserved PEST sequence in ABCA1 (FIG. 1A) was identified. In order to evaluate the role of the PEST sequence, a FLAG-tagged PEST deletion mutant (ABCA1delPEST) was expressed in 293 cells. As shown by biotinylation and immunoprecipitation, cell surface levels of ABCA1delPEST were dramatically increased compared to wild type ABCA1 (FIG. 1B, top, n=5, mean increase=3.9±0.4, p<0.001). In contrast, overall expression levels of ABCAldelPEST in cell lysates were slightly lower than wild type ABCAL (FIG. 1B, bottom). This indicates a marked increase in cell surface concentration of ABCA1delPEST.

To assess the function of ABCA1delPEST, cellular binding of apoA-I and phospholipid and cholesterol efflux to apoA-I was measured. All parameters were increased for the ABCA1delPEST mutant compared to wild type ABCA1 (FIG. 1D, G, H). When normalized for protein expression levels in cell lysates (FIG. 1E), the ABCA1delPEST displayed a remarkable 2-4 fold increase in cholesterol efflux, with the effect being larger at lower DNA transfection levels (FIG. 1F). Thus, deletion of the PEST sequence results in increased cell surface concentration of ABCA1, with a proportionate increase in functional activity. These findings indicate that the PEST sequence has a key role in determining the cell surface expression of functional ABCA1 and suggest that the major active form of ABCA1 resides on the cell surface.

PEST sequences in plasma membrane proteins often increase protein turnover by enhancing the binding of ubiquitin ligases, leading to ubiquitination, endocytosis and intracellular degradation of the target molecule (14). A possible role of the PEST sequence in mediating ubiquitination of ABCAL was initially considered. Using the specific proteasome inhibitor lactacystin, a moderate 2.2-fold increase in total (FIG. 1C) and cell surface ABCA1 levels was found (data not shown) and an increase in the accumulation of total and cell surface ubiquitinated ABCA1 was found. However, these effects were also observed in the ABCA1delPEST version (FIG. 1C). Thus, ubiquitination of ABCA1 is not controlled by the PEST sequence, and inhibition of proteasomal degradation has a smaller effect on ABCA1 cell surface concentration than deletion of the PEST sequence.

In a small number of examples, PEST sequences have been implicated in proteolysis by calpain proteases (13, 15). To see if ABCA1 might be the target of calpain proteases, cells were treated with the specific calpain protease inhibitor, calpeptin. This resulted in a dramatic 4-fold increase (mean increase=3.84±0.32 fold, p<0.001) in ABCA1 protein levels (FIG. 2A). Moreover, the effect of calpeptin was abolished by deletion of the PEST sequence (FIG. 2A). In order to confirm that ABCA1 is a target of calpain protease, cells were permeabilized, washed then treated with purified μ-calpain protease. For wild type ABCA1, this treatment resulted in efficient degradation. However, there was no appreciable degradation of ABCA1delPEST (FIG. 2B). These experiments were conducted in 293 cells transfected with ABCA1. In order to see if calpain protease was also degrading ABCA1 under more physiological conditions, cultures of resident mouse peritoneal macrophages, or primary mouse hepatocytes were treated with calpeptin. This also resulted in an increased ABCA1 protein level (FIG. 2D, E), without any change in ABCA1 mRNA (data not shown). These results indicate that calpain protease is a physiological regulator of ABCA1 protein turnover. Since the effect was observed in several cell types and was reproduced by adding purified u-calpain protease, it is likely to be mediated by calpain 1 and calpain 3, which together form the two subunits of the ubiquitously expressed μ-calpain protease.

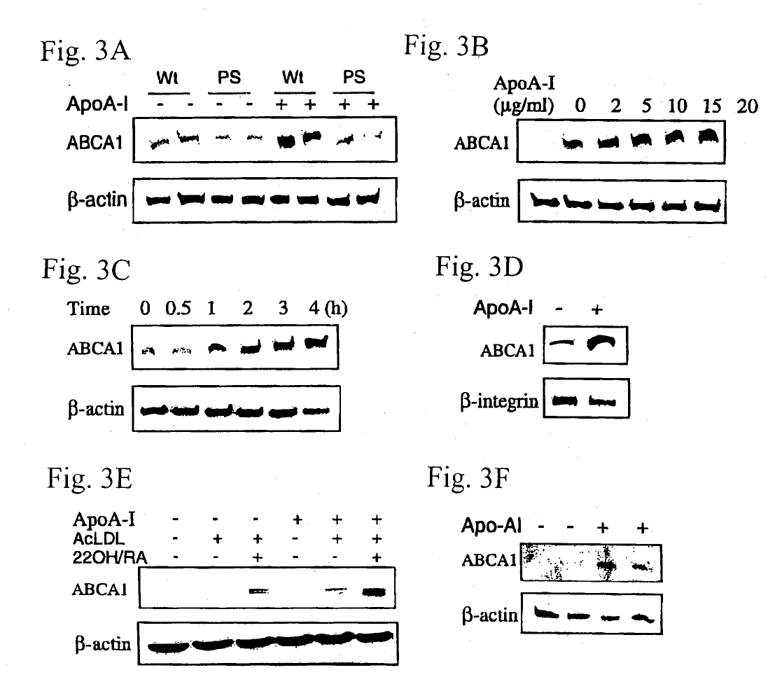

Cellular expression of ABCAL increases binding of apoA-I and apoA-I cross-links to ABCAL (8, 11). Thus, it was hypothesized that apoA-I might modulate the PEST-mediated degradation of ABCA1. Addition of apoA-I to 293 cells, macrophages or hepatocytes resulted in a rapid, marked increase in ABCA1 protein level with effects observed at concentrations of apoA-I likely to be present in biological fluids (FIGS. 3A-C, E), and increased the cell surface expression of ABCA1 (FIG. 3D). In these short-term incubations (<4h), apoA-I did not change ABCA1 mRNA levels (data not shown). In macrophages, the effect of apoA-I required initial up-regulation of ABCA1 gene transcription by cholesterol and oxysterol-loading (FIG. 3B, C), but in hepatocytes the effect of apoA-I was observed without prior sterol loading (FIG. 3E). This is the anticipated result for a post-transcriptional mode of regulation by apoA-I, as hepatocytes appear to use a different ABCA1 promoter than macrophages with higher basal mRNA expression in hepatocytes (16).

Importantly, apoA-I showed these effects for wild type ABCA1 but had no effect on the cellular expression levels of ABCA1delPEST (FIG. 3A), even though apoA-I bound and mediated lipid efflux in cells expressing ABCA1delPEST (FIG. 1). This suggests that the effect of apoA-I on expression of ABCA1 is mediated via the PEST sequence. Moreover, pre-treatment of cells with apoA-I prior to permeabilization abolished the ability of calpain to mediate proteolysis of ABCA1 (FIG. 2C), providing direct evidence linking the effect of apoA-I and calpain-mediated proteolysis. Treatment of cells with apoA-I (with or without proteasome inhibitors) did not decrease ubiquitination levels of ABCA1 (data not shown), excluding decreased proteasomal degradation as a mechanism. These results indicate that the addition of apoA-I to cells activates a positive feedback loop that leads to increased levels of ABCA1 protein. This effect requires the PEST sequence in ABCA1, and is mediated by decreased proteolysis of ABCA1 by calpain protease. Treatment of cells with a fluorescent calpain protease substrate showed no change in degradation upon treatment with apoA-I (data not shown), suggesting that the effect of apoA-I is locally mediated at the plasma membrane. Apparently, this represents the first example of ligand-inhibited calpain protease degradation of a cell surface protein.

The time-course and dose-response of the apoA-I effect on ABCA1 levels (FIG. 3B, C) and on lipid efflux (17) are similar, indicating that lipid efflux could be involved in mediating the effect of apoA-I on ABCAL levels. Thus, experiments were carried out to evaluate the role of cellular phospholipid or cholesterol efflux in this process. Depletion of cellular cholesterol by incubation with cyclodextrin had no effect on cellular ABCA1 levels (FIG. 4A). Also, incubation with HDL2 (50 μg protein/ml), which does not interact with ABCA1 (18), did not affect ABCA1 levels. Mutation of the cytoplasmic ATP-binding Walker motif sequence results in decreased cellular binding of apoA-I and abolishes phospholipid and cholesterol efflux (18, 19). The Walker motif mutant showed increased basal expression and no increase in protein levels when incubated with apoA-I (FIG. 4B). These results could indicate that basal phospholipid translocase activity of ABCA1 is associated with protein degradation and that either binding of apoA-I to ABCA1, or phospholipid efflux mediated by apoA-I provides a signal that leads to decreased ABCA1 degradation.

To distinguish between these possibilities, macrophages were grown in choline-deficient medium, which results in a defect in cellular phospholipid biosynthesis as understood by known methods. This led to decreased phospholipid efflux by apoA-I (data not shown), and abolished the increase in ABCA1 that results from the presence of apoA-I in media (FIG. 4C). However, cellular binding of apoA-I (which is mediated by ABCAL (8)) was actually increased in macrophages grown in choline-free media (FIG. 4D). These results suggest that when phospholipid availability for efflux is limited, apoA-I binds ABCA1 but fails to dissociate, possibly because dissociation requires addition of phospholipid to apoA-I. Together, the data suggest that apoA-I-mediated phospholipid efflux generates a signal that leads to an increase in ABCA1 protein levels. Since apoA-I does not increase the level of ABCA1delPEST, these effects could be mediated via the PEST sequence.

The activity of PEST sequences may be controlled by phosphorylation of S/T residues as this results in increased binding of the calpain calmodulin-like domain (15). Thus, the possibility that apoA-I might alter phosphorylation of ABCA1 was considered. The addition of apoA-I to cells resulted in a time- and dose-dependent reduction in the phosphorylation of ABCA1 (FIG. 5C). After 1-2 h, apoA-I reduced the phosphorylation level of wild type ABCA1 to that seen for ABCA1delPEST (FIG. 5C, D). Compared to wild type ABCA1, the level of phosphorylation was reduced by 70% in ABCA1delPEST (FIG. 5C, D). The PEST sequence of ABCA1 contains a consensus target sequence for casein kinase II (CKII, consensus for CKTI, [ST]-x(2)-[DE]; potential target sequences in PEST, TEDD and SDID). Addition of the CKII inhibitor apigenin was associated with a decrease in phosphorylation of ABCA1 and an increase in ABCA1 protein levels (FIG. 5, other data not shown). Also, the phosphatase inhibitor, okadaic acid, abolished the ability of apoA-I to increase ABCA1 protein levels (FIG. 5). These experiments suggest that the effect of apoA-I on ABCA1 is mediated by a decrease in PEST phosphorylation, related to either decreased kinase or increased phosphatase activity.

The binding of ligands to cell surface receptors or transporters leads to signaling or substrate transport that is often terminated by ubiquitin-mediated endocytosis and degradation in proteasomes or lysosomes (20). While ABCA1 is a target of ubiquitination and proteasomal degradation (FIG. 1C), this is not the primary mode of regulation by apoA-I. This involves an unusual form of regulated proteolysis, involving a PEST sequence in ABCA1, where interaction with the lipid binding ligand (apoA-I) leads to inhibition of calpain-mediated proteolysis and an increase in cell surface ABCA1. This represents a novel form of regulated proteolysis resulting in positive feedback regulation. The effect of apoA-I is likely mediated by decreased phosphorylation of the PEST sequence (FIG. 5). A conformational change of ABCA1 that results from dissociation of phospholipid/apoA-T complexes may alter the accessibility of the PEST sequence to kinase and/or phosphatase, decreasing the phosphorylation of the PEST sequence and thereby inhibiting the binding of the calmodulin-like domain of calpain (15, 21). Phospholipid efflux could also lead to a local change in membrane phospholipids that decreases the binding of a hydrophobic, glycine-rich sequence of the small calpain subunit (22).

ABCA1 expression is highly regulated, both on transcriptional and post-transcriptional levels. The interaction with apoA-I modulates both forms of regulation. Thus, cholesterol efflux promoted by ABCA1 leads to decreased activation of LXR/RXR by oxysterols and ultimately decreases ABCAL transcription and protein levels, but only after cells are no longer cholesterol-loaded. In contradistinction, the present study indicates that phospholipid efflux has a positive effect on ABCA1 protein expression. The positive mode of regulation by apoA-I is likely to be important in hepatocytes and possibly enterocytes. Hepatocytes and enterocytes are continually synthesizing apoA-I that may sustain local ABCA1 protein expression and lead to nascent HDL formation. The availability of free apoA-I at the cell surface may also fluctuate during the day. In the post-prandial state, HDL lipid exchange followed by lipolysis by hepatic lipase may lead to a surge in the availability of free apoA-I and a consequent increase in ABCA1 protein levels. Moreover, although the study was restricted to apoA-I, it is likely that other apoprotein ligands of ABCA1, such as apoE or apoA-IV, will show similar properties (23). Local expression of apoE is induced upon cholesterol loading of macrophages via an LXR mechanism (24), and could result in combined up-regulation of apoe and ABCA1 protein in the foam cell environment.

The mechanisms elucidated herein are rich in therapeutic implications. Infusion or transgenic overexpression of apoA-I or apoe is potently anti-atherogenic (25, 26, 27). This could involve positive feedback control of ABCA1 protein levels, leading to enhanced HDL formation and increased cellular cholesterol efflux. Results indicate a new use for calpain protease inhibitors (28). Also, the local plasma membrane interaction of ABCA1 with calpain protease could be favorably manipulated. Small molecules that inhibit the interaction between calpain and the ABCA1 PEST sequence, by altering phosphorylation or other mechanisms, would be expected to lead to an increase in plasma membrane ABCA1 protein.

### Part II: ApoA-I Infusion Increases ABCA1 In Vivo

In order to determine whether apoA-I could also increase ABCA1 in vivo, mice were injected intravenously with apoA-I. This resulted in an induction of ABCA1 protein in liver and peritoneal macrophages (FIGS. 6A and 6B). The effects in liver were observed in chow-fed animals, whereas those in macrophages were seen after feeding a high-fat, high-cholesterol diet for 7 days. ApoA-I infusion did not significantly alter ABCAL mRNA levels in liver, as determined by quantitative real-time PCR using a TaqMan probe (data not shown). A time-course study of apoA-I infusion showed that the increase in ABCA1 was sustained for 8 hours, but after 24 hours ABCA1 protein levels had returned to base line, likely reflecting the rapid clearance of apoA-I from plasma. Also measured were ABCA1 protein levels in liver from apoA-I transgenic (n=6 mice per group) and apoA-I knockout mice (n=2 mice). No significant difference from controls (for the wild-type mice, mean ABCA1 level normalized to actin=0.25±0.14; for the apoA-I transgenic, mean=0.15±0.03, P=0.18) was found, suggesting that some form of chronic adaptation may occur as a result of continuous expression.

This study reveals a novel mode of regulation of ABCA1 by calpain proteolysis, which is reversed by the extracellular ligand apoA-I acting through a PEST sequence in ABCA1. The findings appear to represent the first example of positive feedback control of a cell surface transporter in which the ligand turns off PEST sequence-regulated calpain proteolysis of the transporter. The nature of the signal that links apolipoprotein binding to proteolysis is unknown, but this process could be initiated by dissociation of phospholipid/apoA-I complexes from the transporter. The increase in ABCA1 in-vivo is consistent with the hypothesis that antiatherogenic effects of apoA-I infusion are mediated by enhanced macrophage cholesterol efflux (30, 31), and indicates that strategies to mimic the effects of apolipoprotein binding, or to inhibit calpain proteolysis, would be expected to increase ABCA1 and decrease atherosclerosis.

The involvement of the PEST sequence in calpain proteolysis and apolipoprotein stabilization of ABCA1 was shown by multiple approaches. First, the major phenotypes produced by deletion of the PEST sequence or calpeptin were similar (i.e., a marked increase in cell surface ABCA1), and there was no additional increase when treatments were combined, indicating that PEST deletion and calpeptin are both acting in the same pathway. Similarly, the effect of apoA-I on ABCA1 levels was blocked by deletion of the PEST sequence. Calpain proteolysis and apolipoprotein effects to the PEST sequence were independently linked by demonstrating that the degradation of ABCA1 by purified u-calpain in permeabilized cells was abolished both by the PEST deletion and by pretreatment with apoA-I. One difference between the phenotype that resulted from calpeptin or apolipoprotein treatment and the phenotype of the PEST deletion mutant was that the latter did not show an increase in total ABCA1 in cell lysates. This probably reflects an additional defect in synthesis of the PEST deletion mutant, perhaps related to a nonspecific effect of the large deletion on translation or mRNA stability. Consistent with this suggestion, several point mutants in the PEST sequence have been found to increase both cell surface and total ABCA1 (data not shown).

Thus, cell surface ABCA1 is regulated by a mechanism that may be more generally relevant to the regulation of cell surface transporters by calpain proteolysis. In the basal state, ABCA1 likely binds calpain via its PEST sequence, resulting in proteolysis. The interaction of ABCA1 with the extracellular, lipid-binding ligand (apoA-I or apoE) leads to inhibition of calpain-mediated proteolysis and an increase in total and cell surface ABCA1. One possible mechanism is that the binding of apoA-I causes a conformational change in ABCA1 that leads to decreased binding of calpain protease to the PEST sequence. Another possibility is that these effects are brought about by phospholipid efflux mediated by apoA-I. Phospholipid efflux mediated by apoA-I might lead to a local change in membrane phospholipids that decreases the binding of a hydrophobic, glycine-rich sequence of the small calpain subunit, which stabilizes binding of calpain to membranes (22). This possibility is favored by a key mutant ABCA1 that binds apoA-I but does not mediate lipid efflux (32). ApoA-I fails to increase ABCA1-W590S (data not shown), implying that phospholipid efflux is required for apoA-I-mediated ABCA1 stabilization. The results suggest that a signaling process may be initiated by apoA-I-mediated phospholipid efflux, leading to decreased binding of calpain to the PEST sequence.

Calpain proteases comprise a small gene family with 12 identified members (33). Since the inhibition of ABCA1 degradation by calpeptin was observed in several cell types and was reproduced by adding purified u-calpain protease, it is likely to be mediated by capn1 and capn4, which together form the two subunits of the widely expressed μ-calpain protease (36). However, the possible involvement of more specific calpains in different cell types cannot be ruled out.

While these studies were ongoing, Arakawa and Yokoyama reported the apolipoprotein stabilization of ABCA1 in cell culture (34), and the above major findings are consistent with and complementary to theirs. The subject study was initially focused on the role of a PEST sequence in regulating calpain proteolysis of ABCA1 and provides an important link with the earlier work (34) by showing that the apolipoprotein effects are mediated by inhibition of calpain proteolysis and require the PEST sequence. Moreover, the stabilization of ABCA1 by apoA-I and apoE in primary macrophage cultures is shown, as is the likely involvement of similar mechanisms for both apolipoproteins have been shown.

In contrast to an earlier study (34), it was shown that ABCA1 is ubiquitinated and that lactacystin increased ABCA1 in transfected 293 cells and acetyl-LDL-loaded, LXR/RXR-activated macrophages (data not shown). However, the effect of lactacystin was moderate and irrelevant to the PEST sequence-mediated calpain degradation. Moreover, expression of the N-terminal half of ABCA1, which does not contain the PEST sequence, also resulted in cell surface expression and ubiquitination (not shown), suggesting that the ubiquitinated residues are remote from the PEST sequence.

Together, the instant studies indicate that under normal cellular conditions, ABCA1 turnover is rapid, probably primarily reflecting nonproteasomal calpain-mediated degradation. Similar to these findings, Feng and Tabas (35) found that lactacystin increased ABCA1 in macrophages loaded with acetyl-LDL and cholesterol ester. However, the effect of lactacystin became more pronounced in macrophages loaded with acetyl-LDL in the presence of an acyl-CoA:cholesterol acyltransferase inhibitor, leading to the suggestion that the proteasomal degradation pathway is activated in free cholesterol-loaded cells (35). Together these studies suggest that a distinct ubiquitin-proteasome degradation pathway of ABCA1 is activated in free cholesterol loaded macrophages, a condition that eventuates in cell death (36). Thus, two different ABCA1 degradation pathways can be active under various cellular conditions: a basal calpain degradation pathway that is turned off by interaction with apolipoproteins, and a ubiquitin-proteasome pathway that is activated by marked free-cholesterol loading. There is a precedent for such dual regulation in the distinct proteasome and calpain degradation pathways of IB (15).

Importantly, it is demonstrated that ABCA1 is increased in hepatocytes and macrophages in vivo following apoA-I infusion. It could appear paradoxical that injection of apoA-I in an amount representing approximately 15% of total plasma apoA-I induces an increase in macrophage and hepatic ABCA1. However, the majority of apoA-I associated with bulk HDL is a poor substrate of ABCA1 compared with lipid-poor apoA-I (18). Injection of free apoA-I is expected to substantially increase the latter pool in plasma.

The findings of these in vivo studies help explain the antiatherogenic effects of apoA-I and apoe infusion and help provide a rationale for apoA-I infusion trials in humans. Intravenous injection of apoA-I increased ABCA1 in liver and peritoneal macrophages. The increase in ABCA1 in macrophages is likely to lead to an increase in macrophage cholesterol efflux and to reversal of foam cell formation. While macrophage ABCA1 does not make a major contribution to plasma HDL levels (37), the stabilization of ABCA1 in liver is likely to result in an increase in HDL levels. Increased HDL levels could promote macrophage cholesterol efflux by multiple mechanisms and could also be antiatherogenic through anti-inflammatory effects of HDL (3). Unexpectedly, increases in ABCA1 protein were not found in apoA-I transgenic mice; this highlights the complexity of these models, and the likely existence of multiple antiatherogenic mechanisms operating through the HDL fraction.

ABCA1 expression is highly regulated, on both transcriptional and posttranscriptional levels. The interaction with apoA-I modulates both forms of regulation. Thus, cholesterol efflux promoted by ABCA1 leads to decreased activation of LXR/RXR by oxysterols and ultimately decreases ABCA1 transcription and protein levels, but only after cells are no longer cholesterol-loaded. In contrast, the present study indicates that apoA-I and apoe have a positive effect on ABCA1 protein expression, and this is likely to be important in hepatocytes and macrophage foam cells. Intense interest has recently centered on the possibility that increasing macrophage cholesterol efflux could represent a novel approach to treatment of atherosclerosis (3). LXR/RXR targets a battery of genes that mediate cholesterol efflux, transport, and excretion, and LXR activators are antiatherogenic (38). However, LXR/RXR also increases transcription of SREBP1c and its target genes, causing fatty liver and hypertriglyceridemia (39, 40). The instant results indicate that calpain protease inhibitors (28), or small molecules that modulate the local interaction of ABCA1 with calpain protease at the plasma membrane, would provide an alternative way to upregulate ABCA1 protein. This strategy is especially appealing since it could mimic the stabilizing effect of the natural ligands apoA-I and apoE.

Materials and Methods

Chemicals and reagents: Human apoA-I (BIODESIGN International, Saco, Me., USA) was dialyzed against PBS. PD8407 anti-ubiquitin antibody was from Santa Cruz Biotechnology Inc. (Santa Cruz, Calif., USA); rabbit polyclonal anti-ABCA1 antibody was from Novus Biologicals Inc. (Littleton, Colo., USA). Purified μ-calpain, calpeptin, lactacystin, and N-Ac-Leu-Leu-norleucinal (ALLN) were from Calbiochem-Novabiochem Corp. (San Diego, Calif., USA).

Plasmid constructs and cell transfection: ABCA1-FLAG was constructed as described (18). ABCA1delPEST was based on ABCA1-FLAG. Using PCR, ABCA1delPEST was constructed by deleting a nucleotide sequence that encodes mouse ABCA1 amino acids 1283-1306 and was confirmed by sequencing. HEK293 cells, in 12- or 24-well collagen-coated plates, were transiently transfected with various plasmid constructs at indicated DNA concentrations with Lipofectamine 2000 (Invitrogen Corp., San Diego, Calif., USA) at 37° C. overnight (20 hours). A construct expressing green fluorescence protein (GFP) was routinely used to visually monitor transfection efficiency (i.e., the percentage of cells expressing GFP). The transfection efficiency of 293 cells was in the range of 50-80% of cells. Although transfection efficiency did vary from experiment to experiment, it was found that the variation within the same experiment was small (generally less than 10%). In addition to performing multiple replicates within each experiment, all experiments were repeated on multiple separate occasions to confirm reproducibility of results.

Cellular lipid efflux assays, apoA-I cell association, and chemical cross-linking: The assays were carried out as in (14). Generally, 293 cells were labeled by culturing overnight in media containing either [³H]cholesterol, for cholesterol efflux, or [³H]choline, for phospholipid efflux. The next day, cells were washed with fresh media before or after treatment as indicated, and then apoA-I was added as acceptor and incubated for the indicated period before the media and cells were collected for analysis. Mouse peritoneal macrophage cells were isolated from male mice by peritoneal lavage with PBS 3 days after intraperitoneal injection with 1 ml of 3.85% thioglycollate. The cells were labeled with 1 mCi/ml [³H]cholesterol overnight in DMEM and 0.2% BSA supplemented with 50 μg/ml acetylated LDL plus LXR/RXR ligands 22(R)-hydroxycholesterol and 9-cis retinoic acid (both 10 μM). After labeling, cells were washed and efflux was carried out with 10 μg/ml apoA-I for 3 hours. Then, cells and medium were collected for analysis. Cholesterol efflux was expressed as the percentage of the radioactivity released from the cells into the medium, relative to the total radioactivity in cells plus medium. For apoA-I cell association, cells were incubated with 0.2 μg/ml [¹²⁵ I]apoA-I in 0.2% BSA and DMEM for 1 hour at 37° C. After being washed three times with fresh media, cells were lysed with 0.1% SDS and 0.1N NaOH lysis buffer, and radioactivity was determined by a gamma counter.

Immunoprecipitation and immunoblot analysis of ABCA1: For immunoblot analysis of ABCA1, ABCA1-FLAG, and ABCA1delPEST-FLAG, transfected HEK293 cells, peritoneal macrophages, or primary hepatocytes were washed and scraped in PBS and lysed in RIPA buffer (10 mM Tris-HCl [pH 7.3], 1 mM MgCl₂, 1.0% Nonidet P-40, 0.5% sodium deoxycholate, and 5 mM EDTA in the presence of protease inhibitors as follows: 0.5 μg/ml leupeptin, 1 μg/ml aprotinin, and 1 μg/ml pepstatin A) (Roche Molecular Biochemicals, Indianapolis, Ind., USA). Postnuclear supernatants containing the indicated amounts of protein were subjected to Western analysis using an anti-ABCA1 antiserum or anti-FLAG M2 antibody and chemiluminescence detection. The relative intensities of the bands were determined by densitometry. For cell surface ABCA1 analysis, cells were first biotinylated with 0.5 mg/ml EZ-Link Sulfo-NHS-SS-Biotin (Pierce Chemical Co., Rockford, Ill., USA) at 4° C. for 30 minutes. Then cells were lysed with RIPA buffer at 4° C. After centrifugation, the supernatant of cell lysates was incubated with anti-FLAG agarose beads overnight at 4° C. Following centrifugation and washing, the collected agarose beads were subjected to SDS-PAGE sample buffer with 100 mM 2-mercaptoethanol. The total or ubiquitinated ABCA1 was detected by Western blot using either anti-FLAG antibody or streptavidin-horseradish peroxidase.

Calpain-catalyzed proteolysis of metabolically labeled ABCA1: Transiently transfected 293 cells were pulse-labeled by [³⁵S]methionine (0.5 mCi/ml) in 0.2% BSA and DMEM for 2 hours. Cells were washed three times with fresh media and placed on ice for 10 minutes. Then cells were permeabilized by addition of 80 μg/ml digitonin in DMEM and incubated on ice for 15 minutes. Next, the cells were washed twice with fresh DMEM, and then purified μ-calpain in DMEM plus 2 mM CaCl₂ was added at the indicated concentration and incubated for 20 minutes at room temperature. Then cells were lysed by addition of 1 ml RIPA buffer with 40 μg/ml calpeptin. FLAG-tagged ABCA1 was immunoprecipitated by anti-FLAG agarose beads and subjected to quantitative analysis by PhosphorImager.

Primary hepatocytes: Hepatocytes were isolated according to Honkakoski and Negishi (29), except that complete protease inhibitor was added to digestion buffer according to the manufacturer's instructions (Roche Molecular Biochemicals).

Apolipoprotein A-I infusion in mice: All mice used in these studies were 8-week-old female wild-type mice of the inbred strain C57BL/6J and were fed a chow diet. Mice were anesthetized intraperitoneally with 0.1 ml/30 g body weight of a solution containing 100 mg/ml ketamine and 30 mg/ml xylazine. ApoA-I (20 mg/kg body weight) or BSA as control was administered via femoral vein as a bolus injection. Four hours after injection, animals were euthanized and the liver was dissected out for analysis.

### REFERENCES

1. M. S. Brown, Y. K. Ho, J. L. Goldstein, J Biol Chem 255, 9344-52 (1980).

2. A. R. Tall, J Intern Med 237, 5-12 (1995).

3. A. J. Lusis, Nature 407, 233-41 (2000).

4. E. J. Schaefer, L. A. Zech, D. E. Schwartz, H. B.

Brewer, Jr., Ann Intern Med 93, 261-6 (1980).

5. A. Brooks-Wilson, et al., Nat Genet 22, 336-45 (1999).

6. M. Bodzioch, et al., Nat Genet 22, 347-51 (1999).

7. S. Rust, et al., Nat Genet 22, 352-5 (1999).

8. N. Wang, D. L. Silver, P. Costet, A. R. Tall, J Biol Chem 275, 33053-8 (2000).

9. J. J. Repa, et al., Science 289, 1524-9 (2000).

10. P. Costet, Y. Luo, N. Wang, A. R. Tall, J Biol Chem 275, 28240-5 (2000).

11. J. F. Oram, R. M. Lawn, M. R. Garvin, D. P. Wade, J Biol Chem 275, 34508-11 (2000).

12. T. Langmann, et al., Biochem Biophys Res Commun 257, 29-33 (1999).

13. M. Rechsteiner, S. W. Rogers, Trends Biochem Sci 21, 267-71 (1996).

14. A. F. Roth, D. M. Sullivan, N. G. Davis, J Cell Biol 142, 949-61 (1998).

15. S. D. Shumway, M. Maki, S. Miyamoto, J Biol Chem 274, 30874-81 (1999).

16. L. B. Cavelier, et al., J Biol Chem 276, 18046-51 (2001).

17. W. Chen, et al., J Biol Chem 276, 43564-9 (2001).

18. N. Wang, D. L. Silver, C. Thiele, A. R. Tall, J Biol Chem 276, 23742-7 (2001).

19. D. Marguet, M. F. Luciani, A. Moynault, P. Williamson, G. Chimini, Nat Cell Biol 1, 454-6 (1999).

20. S. K. Shenoy, P. H. McDonald, T. A. Kohout, R. J. Lefkowitz, Science 294, 1307-13 (2001).

21. J. Shen, P. Channavajhala, D.C. Seldin, G. E. Sonenshein, J Immunol 167, 4919-25 (2001).

22. S. Imajoh, H. Kawasaki, K. Suzuki, J Biochem (Tokyo) 99, 1281-4 (1986).

23. A. T. Remaley, et al., Biochem Biophys Res Commun 280, 818-23 (2001).

24. B. A. Laffitte, et al., Proc Natl Acad Sci USA 98, 507-12 (2001).

25. A. Miyazaki, et al., Arterioscler Thromb Vasc Biol 15, 1882-8 (1995).

26. N. Yamada, et al., J Clin Invest 89, 706-11 (1992).

27. A. S. Plump, C. J. Scott, J. L. Breslow, Proc Natl Acad Sci USA 91, 9607-11 (1994).

28. A. Stracher, Ann N Y Acad Sci 884, 52-9 (1999).

29. Honkakoski, P., and Negishi, M., Biochem. J. 330:889-95 (1998).

30. Chiesa, G. et al. Circ. Res. 90:974-980 (2002).

31. Eriksson, M., Carlson, L. A., Miettinen, T. A., and Angelin, B. Circulation. 100:594-598 (1999).

32. Fitzgerald, M. L. et al., J. Biol. Chem. 277:33178-33187 (2002).

33. Sorimachi, H., and Suzuki, K., J. Biochem. (Tokyo). 129:653-664 (2001).

34. Arakawa, R., and Yokoyama, S., J. Biol. Chem. 277:22426-22429 (2002).

35. Feng, B., and Tabas, I., J. Biol. Chem. 277:43271-43280 (2002).

36. Yao, P. M., and Tabas, I, J. Biol. Chem. 275:23807-23813 (2000).

37. Haghpassand, M., Bourassa, P. A., Francone, O. L., and Aiello, R. J., J. Clin. Invest. 108:1315-1320 (2001).

38. Joseph, S. B. et al., Proc. Natl. Acad. Sci. USA. 99:7604-7609 (2002).

39. Repa, J. J. et al., Genes Dev. 14:2819-2830 (2000).

40. Schultz, J. R. et al., Genes Dev. 14:2831-2838 (2000).

41. Wang, N., Silver, D. L., Thiele, C., and Tall, A. R., J. Biol. Chem. 276:23742-23747 (2001).

What is claimed is:

1. An isolated ABCA1 protein having a mutation within its PEST domain.

2. The protein of claim 1, wherein the protein is a human protein.

3. The protein of claim 1, wherein the mutation is selected from the group consisting of a point mutation, an insertion mutation and a deletion mutation.

4. The protein of claim 3, wherein the mutation is a deletion mutation.

5. An isolated nucleic acid encoding an ABCA1 protein having a mutation within its PEST domain.

6. The nucleic acid of claim 5, wherein the nucleic acid is DNA or RNA.

7. The nucleic acid of claim 6, wherein the nucleic acid is DNA.

8. A vector comprising the nucleic acid of claim 5.

9. The vector of claim 8, wherein the vector is an expression vector.

10. A host-vector system comprising a host cell having therein an expression vector comprising a nucleic acid encoding an ABCA1 protein having a mutation within its PEST domain.

11. The host-vector system of claim 10, wherein the cell is a prokaryotic cell or a eukaryotic cell.

12. The host-vector system of claim 11, wherein the cell is a mammalian cell.

13. An isolated cell comprising an ABCA1 protein having a mutation within its PEST domain, which protein has a decreased rate of turnover in the cell.

14. A method for determining whether a protease inhibitor reduces the turnover rate of ABCA1 protein in a cell, comprising the steps of
(a) contacting the cell with the protease inhibitor under physiological conditions;
(b) determining the rate of turnover of ABCA1 protein turnover in the cell; and
(c) comparing the rate of turnover so determined with a known standard, thereby determining whether the protease inhibitor reduces the ABCA1 protein turnover rate in the cell.

15. A method for determining whether a protease inhibitor increases the rate of cholesterol efflux from a cell, comprising the steps of
(a) contacting the cell with the protease inhibitor under physiological conditions;
(b) determining the rate of cholesterol efflux from the cell; and
(c) comparing the rate of cholesterol efflux so determined with a known standard, thereby determining whether the protease inhibitor increases the rate of cholesterol efflux from the cell.

16. The method of claim 14 or 15, wherein the cell is a human cell.

17. The method of claim 14 or 15, wherein the cell is a macrophage.

18. The method of claim 14 or 15, wherein the protease inhibitor is a calpain inhibitor.

19. A method for increasing ABCA1-mediated cholesterol efflux from a cell comprising the step of inhibiting the PEST-mediated proteolysis of ABCA1 protein in the cell.

20. The method of claim 19, wherein the cell is a human cell.

21. The method of claim 19, wherein the cell is a macrophage.

22. The method of claim 19, wherein the inhibiting comprises contacting the cell with a calpain inhibitor.

23. A method for treating a subject afflicted with atherosclerosis comprising administering to the subject an agent that inhibits the PEST-mediated proteolysis of ABCA1 protein in the subject's macrophages.

24. The method of claim 23, wherein treating the subject comprises stabilizing arterial plaque in the subject.

25. A method for increasing the formation of HDL in a subject comprising administering to the subject an agent that inhibits the PEST-mediated proteolysis of ABCA1 protein in the subject's cells.

26. The method of claim 23, wherein the subject is a human.

27. The method of claim 23, wherein the agent is a protease inhibitor.

28. The method of claim 27, wherein the protease inhibitor is a calpain inhibitor.

29. A pharmaceutical composition comprising an agent that inhibits the PEST-mediated proteolysis of ABCA1 protein in a cell, and a pharmaceutically acceptable carrier.

30. An article of manufacture comprising a packaging and a pharmaceutical agent, wherein (a) the pharmaceutical agent inhibits the PEST-mediated proteolysis of ABCA1 protein in a cell, and (b) the packaging comprises a label indicating the use of the agent for treating atherosclerosis in a subject.

31. The pharmaceutical composition of claim 29 or the article of claim 30, wherein the agent is a protease inhibitor.

32. The pharmaceutical composition of claim 29 or article of claim 30, wherein the agent is a calpain inhibitor.

33. The pharmaceutical composition of claim 29 or the article of claim 30, wherein the subject is a human.
